(19) United States
(12) Patent Application Publication
(10) Pub. No.: US 2023/0233427 A1
(43) Pub. Date: Jul. 27, 2023

(54) COLOR-STABLE SUNSCREEN

(71) Applicant: BEIERSDORF AG, Hamburg (DE)

(72) Inventors: Alexis GOULET-HANSSENS, Hamburg (DE); Jan NILSSON, Hamburg (DE); Christoph ERNST, Hamburg (DE); Ana Laura GARCIA REYES, Hamburg (DE); Magdalena VON WEDEL-PARLOW, Hamburg (DE)

(21) Appl. No.: 17/996,641

(22) PCT Filed: Mar. 22, 2021
(86) PCT No.: PCT/EP2021/057288

§ 371 Date: Oct. 20, 2022

(30) Foreign Application Priority Data
Apr. 21, 2020 (DE) 10 2020 205 042.2

(51) Int. Cl. A61K 8/35; A61Q 17/04; A61K 8/41; A61K 8/37; A61K 8/19

(52) U.S. Cl. CPC A61K 8/35; A61K 8/19; A61K 8/37; A61K 8/415; A61Q 17/04; A61K 2800/30; A61K 2800/43; A61K 2800/5922

(57) ABSTRACT

Cosmetic preparation containing a) 4-(tert.-butyl)-4′-methoxydibenzoyl methane and/or (2-[-4-(diethylamino)-2-hydroxybenzoyl] benzoic acid hexyl ester (INCI: Diethylamino Hydroxybenzoyl Hexyl Benzoate) and b) diisostearoyl polyglyceryl-3-dimer-dilinoleate.

The present invention relates to a cosmetic preparation comprising 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate), and Diisostearoyl Polyglyceryl-3 Dimer Dilinoleate, to a process for producing color-stable cosmetic preparations comprising 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate), characterized in that Diisostearoyl Polyglyceryl-3 Dimer Dilinoleate is added to the preparation, and to the use of Diisostearoyl Polyglyceryl-3 Dimer Dilinoleate in cosmetic preparations comprising 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate) for preventing the formation of complexes with metal ions.

For many years there has been an unbroken trend away from aristocratic pallor toward “healthy, athletically brown skin”. To achieve this, people expose their skin to solar radiation, since this causes pigment formation in the sense of melanin formation. However, the ultraviolet radiation of sunlight also has a harmful effect on the skin. Besides acute damage (sunburn), long-term damage such as an increased risk of developing skin cancer occurs in the case of excessive irradiation with light from the UVB range (wavelength: 280-320 nm). Excessive exposure to UVB and UVA radiation (wavelength: 320-400 nm) additionally leads to weakening of the elastic and collagen fibers in connective tissue. This leads to numerous phototoxic and photoallergic reactions, resulting in premature aging of the skin.

Therefore, to protect the skin, a range of photoprotective filter substances that can be used in cosmetic preparations have been developed. In most industrialized countries, these UVA and UVB filters are collated in the form of positive lists such as Annex 7 of the Kosmetikverordnung [German Cosmetics Ordinance].

The multiplicity of commercially available sunscreens must not, however, distract from the fact that these prior art preparations have a range of disadvantages.

Cosmetic preparations and particularly sunscreen preparations usually contain complexing agents, with use preferably being made of EDTA and salts thereof (in particular sodium salts). These serve to complex the metal ions, such as iron or aluminum ions, that have been introduced by water (as an ingredient of numerous preparations), further ingredients (for example color pigments) or the packaging, since said metal ions can destabilize and discolor the preparation. Particularly in the case of preparations comprising the UV-A filters 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate) that form reddish-colored metal complexes, such an addition according to the prior art is absolutely required since otherwise the preparation would have a reddish-brown color. These effects are described, for example, in DE 19853288.

A disadvantage of the prior art is the fact that the use of EDTA and salts thereof is being regarded increasingly critically by some of those skilled in the art. There is a concern that this compound might not be sufficiently rapidly biodegradable, or that it might promote skin penetration. Whether or not such concerns are justified can be left open within the scope of the present disclosure and be left to the further investigations by those skilled in the art. The fact, however, is that consumers are increasingly wanting products that contain as little EDTA as possible, and ideally none.

The problem was therefore to develop a cosmetic preparation (in particular a sunscreen) that contains as little EDTA as possible and is nevertheless color-stable and insensitive to metal ions.

The problem is surprisingly solved by a cosmetic preparation containing
a) 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate) and
b) Diisostearoyl Polyglyceryl-3 Dimer Dilinoleate.

The problem is surprisingly further solved by a process for producing color-stable cosmetic preparations comprising 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate), characterized in that Diisostearoyl Polyglyceryl-3 Dimer Dilinoleate is added to the preparation.

Last but not least, the problem is surprisingly solved by the use of Diisostearoyl Polyglyceryl-3 Dimer Dilinoleate in cosmetic preparations comprising 4-(tert-butyl)-4′-methoxydibenzoylmethane and/or hexyl 2-[4-(diethylamino)-2-hydroxybenzoyl]benzoate (INCI: Diethylamino Hydroxybenzoyl Hexyl Benzoate) for preventing the formation of complexes with metal ions.

According to the invention, metal ions are understood to mean aluminum, iron and copper ions.

Within the scope of the present disclosure, the phrases “according to the invention”, “advantageous according to the invention” etc. always relate to the cosmetic preparation (sunscreen) according to the invention, the uses according to the invention and the process according to the invention, unless otherwise described in the individual case.

Although the prior art includes DE 102014202377.7, this document was unable to point the way to the present invention.

It is advantageous according to the invention if the preparation is free of EDTA.

Furthermore, it is advantageous according to the invention if the preparation is free of 3-(4-methylbenzylidene)camphor, 2-hydroxy-4-methoxybenzophenone (INCI: Oxybenzone), 2-ethylhexyl 4-methoxycinnamate (INCI: Octyl Methoxycinnamate), parabens (particularly methyl, propyl and butyl paraben), methylisothiazolinone, chloromethylisothiazolinone and DMDM hydantoin, polyethylene glycol ethers or polyethylene glycol esters.

Embodiments of the present invention that are advantageous according to the invention are further characterized in that the preparation is free of octocrylene.

It is advantageous in the sense of the present invention if the preparation contains Diisostearoyl Polyglyceryl-3 Dimer Dilinoleate which is characterized in that it has a viscosity of greater than/equal to 1500 mPas in a 50% by weight solution in C12-15 alkyl benzoate at 25° C. According to the invention, such a solution preferably has a viscosity of greater than/equal to 3000 mPas and particularly preferably a viscosity of greater than/equal to 3400 mPas.

Alternatively, this measurement can also be performed in 50% Caprylic/Capric Triglyceride solution.

This viscosity is determined according to the invention as follows: All values relate to The measurements are performed at 25° C. in a 150 ml rolled rim glass bottle using the Rheomat R 123 rotational viscometer from proRheo. The measuring bob used is the No.1 measuring bob (Article No. 200 0191). It speed range 62.5 min⁻¹ uses. If no other information is given, then the viscosity is always measured 24 h after the mixture is produced.

The same applies to all viscosity values specified, which always relate to a measurement 24 h after production, unless a different time is specified.

It is advantageous according to the invention if the preparation contains Diisostearoyl Polyglyceryl-3 Dimer Dilinoleate at a concentration of 0.1% to 5% by weight, based on the total weight of the preparation. Preferred according to the invention here is a concentration of 0.5% to 3% by weight, based on the total weight of the preparation.

Three embodiments of the present invention that are advantageous according to the invention can be distinguished:

If the preparation contains 4-(tert-butyl)-4′-methoxydibenzoylmethane and no hexyl 2-[4-(diethylamino)-2-hydroxybenzoyl]benzoate (INCI: Diethylamino Hydroxybenzoyl Hexyl Benzoate), then it is advantageous according to the invention if the preparation contains 4-(tert-butyl)-4′-methoxydibenzoylmethane at a concentration of 0.1% to 5% by weight, based on the total weight of the preparation, with a concentration of 1% to 5% by weight, based on the total weight of the preparation, being preferred according to the invention.

The weight ratio of 4-(tert-butyl)-4′-methoxydibenzoylmethane to Diisostearoyl Polyglyceryl-3 Dimer Dilinoleate in the preparation, advantageously according to the invention, is 1:50 to 1:1.

If the preparation contains 2-[4-(diethylamino)-2-hydroxybenzoyl]benzoate (INCI: Diethylamino Hydroxybenzoyl Hexyl Benzoate) and no 4-(tert-butyl)-4′-methoxydibenzoylmethane, then it is advantageous according to the invention if the preparation contains Diethylamino Hydroxybenzoyl Hexyl Benzoate at a concentration of 0.1% to 10% by weight, based on the total weight of the preparation, with a concentration of 0.5% to 8% by weight, based on the total weight of the preparation, being preferred according to the invention.

The weight ratio of Diethylamino Hydroxybenzoyl Hexyl Benzoate to Diisostearoyl Polyglyceryl-3 Dimer Dilinoleate in the preparation, advantageously according to the invention, is 1:50 to 2:1.

If the preparation contains 4-(tert-butyl)-4′-methoxydibenzoylmethane and hexyl 2-[4-(diethylamino)-2-hydroxybenzoyl]benzoate (INCI: Diethylamino Hydroxybenzoyl Hexyl Benzoate), then it is advantageous according to the invention if the preparation contains 4-(tert-butyl)-4′-methoxydibenzoylmethane at a concentration of 0.1% to 5% by weight, based on the total weight of the preparation, with a concentration of 1% to 5% by weight, based on the total weight of the preparation, being preferred according to the invention.

Diethylamino Hydroxybenzoyl Hexyl Benzoate is used in this case at a concentration of 0.1% to 10% by weight, based on the total weight of the preparation, with a concentration of 1% to 10% by weight, based on the total weight of the preparation, being preferred according to the invention.

The weight ratio of the total amount of 4-(tert-butyl)-4′-methoxydibenzoylmethane and hexyl 2-[4-(diethylamino)-2-hydroxybenzoyl]benzoate (INCI: Diethylamino Hydroxybenzoyl Hexyl Benzoate) to Diisostearoyl Polyglyceryl-3 Dimer Dilinoleate, advantageously according to the invention, is 1:25 to 3:1.

Embodiments of the present invention that are advantageous according to the invention are characterized in that the preparation contains iron-containing color pigments.

By way of example, the following may be used as iron-containing color pigments: Unipure Red LC 386, Unipure Brown LC 889, Unipure Brown LC 881, Unipure Yellow LC 188, Unipure Black LC 989 (from Sensient Cosmetic Technologies), BLACK NF (from Kobo Products, Inc.).

The preparation according to the invention may be in the form of an aqueous-alcoholic solution, of an alcoholic solution, of a hydrodispersion, of a W/O emulsion or of an O/W emulsion; those skilled in the art will distinguish a hydrodispersion from an O/W emulsion on the basis of the emulsifier content. It is advantageous according to the invention if the preparation is in the form of an O/W emulsion.

In such a case, it is advantageous according to the invention if the preparation contains one or more O/W emulsifiers selected from the group of compounds glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate.

Embodiments of the present invention that are advantageous according to the invention are further characterized in that the preparation contains 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), 2,4,6-tris[anilino(p-carbo-2′-ethyl-1′-hexyloxy)]-1,3,5-triazine (INCI: Ethylhexyl Triazone) and/or 2-phenylbenzimidazole-5-sulfonic acid salts.

If the preparation contains 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), then the use concentration that is advantageous according to the invention is from 0.5% to 4.5% by weight based on the total weight of the preparation.

If the preparation contains 2,4,6-tris[anilino(p-carbo-2′-ethyl-1′-hexyloxy)]-1,3,5-triazine (INCI: Ethylhexyl Triazone), then the use concentration that is advantageous according to the invention is 0.5% to 10% by weight based on the total weight of the preparation.

If the preparation contains 2-phenylbenzimidazole-5-sulfonic acid salts (the sodium salt preferably being used), then the use concentration that is advantageous according to the invention is 0.1% to 3% by weight based on the total weight of the preparation.

Furthermore, the preparation according to the invention may contain further UV filters.

It is advantageous according to the invention if the preparation is free of xanthan gum (INCI: Xanthan Gum).

In such a case, the embodiments that are preferred according to the invention are characterized in that the preparation contains xanthan gum (INCI: Xanthan Gum) at a concentration of 0.01% to 1% by weight, based on the total weight of the preparation.

Advantageous embodiments in the sense of the present invention are characterized in that the preparation contains phenoxyethanol, ethylhexylglycerin and/or 4-hydroxyacetophenone.

It is further advantageous according to the invention if the preparation contains one or more alkanediols from the following group of compounds: pentane-1,2-diol, hexane-1,2-diol, octane-1,2-diol, decane-1,2-diol, 2-methylpropane-1,3-diol.

Embodiments of the present invention that are advantageous according to the invention are characterized in that the preparation contains one or more compounds selected from the group of compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, thiamidol, 8-hexadecene-1,16-dicarboxylic acid, glycerylglucose, (2-hydroxyethyl)urea, vitamin E and derivatives thereof, hyaluronic acid and/or salts thereof, and/or licochalcone A.

The preparation according to the invention may advantageously contain humectants. Humectants (moisturizers) are substances or substance mixtures which give cosmetic preparations the property, once applied to or spread on the surface of the skin, of reducing the loss of moisture from the horny layer (also known as transepidermal water loss (TEWL)) and/or of having a beneficial effect on the hydration of the horny layer.

Advantageous humectants (moisturizers) in the sense of the present invention are, for example, glycerin, lactic acid and/or lactates, in particular sodium lactate, butylene glycol, propylene glycol, Biosaccharide Gum-1, glycine soja, ethylhexyloxyglycerin, pyrrolidonecarboxylic acid and urea. In addition, it is especially advantageous to use polymeric moisturizers from the group of the water-soluble and/or water-swellable polysaccharides and/or those that can be gelated with the aid of water. Especially advantageous are, for example, hyaluronic acid, chitosan and/or a fucose-rich polysaccharide which is registered in Chemical Abstracts under the registration number 178463-23-5 and is available, for example, under the Fucogel®1000 name from SOLABIA S.A. Moisturizers can advantageously also be used as anti-wrinkle agents for protecting against skin changes of the kind for example that occur when the skin ages.

The cosmetic preparations according to the invention may also advantageously, although not mandatorily, contain fillers which further improve, for example, the sensory and cosmetic properties of the formulations and generate or enhance, for example, a velvety or silky skin feel. Advantageous fillers in the sense of the present invention are starch and starch derivatives (such as tapioca starch, distarch phosphate, aluminum or sodium starch octenylsuccinate and the like), pigments which principally have neither UV filter nor coloring effects (such as boron nitride etc.), and/or Aerosils^{®} (CAS No. 7631-86-9) and/or talc and/or polyethylene, nylon.

It is preferred according to the invention if the preparation according to the invention contains tapioca starch and/or distarch phosphate.

Embodiments of the present invention that are advantageous according to the invention are characterized in that the preparation contains one or more oils selected from the group of compounds Butylene Glycol Dicaprylate/Dicaprate, Phenethyl Benzoate, C12-15 Alkyl Benzoate, dibutyl adipate, diisopropyl sebacate, dicaprylyl carbonate, Di-C12-13 Alkyl Tartrate, Butyloctyl Salicylate, Diethylhexyl Syringylidene Malonate, Hydrogenated Castor oil Dimerate, Triheptanoin, C12-13 Alkyl Lactate, C16-17 Alkyl Benzoate, Propylheptyl Caprylate, Caprylic/Capric Triglyceride, Diethylhexyl 2,6-Naphthalate, Octyldodecanol, Caprylic/Capric Triglyceride, Ethylhexyl Cocoate.

It is preferred according to the invention here if the preparation contains dibutyl adipate, dicaprylyl carbonate, Butylene Glycol Dicaprylate/Dicaprate and/or C12-C15 alkyl benzoate.

The water phase of the preparations according to the invention may advantageously contain customary cosmetic auxiliaries, such as alcohols, particularly those of low carbon number, preferably ethanol and/or isopropanol, or polyols of low carbon number and ethers thereof, preferably propylene glycol, glycerin, electrolytes, self-tanning agents, and particularly one or more thickeners which may advantageously be selected from the following group: silicon dioxide, aluminum silicates, polysaccharides and derivatives thereof, for example hyaluronic acid, hydroxypropyl methylcellulose.

It is advantageous according to the invention if the preparation according to the invention contains perfumes.

It is preferred according to the invention here if the total concentration of perfumes in the preparation is from 0.00001% to 1% by weight, based on the total weight of the preparation.

It is particularly preferred according to the invention if the total concentration of perfumes in the preparation is from 0.00005% to 0.5% by weight, based on the total weight of the preparation.

It is advantageous in the sense of the present invention here if the preparation according to the invention contains one or more perfumes selected from the group of compounds limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methylionone, amyl C, butylphenyl methylpropional cinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, Evernia furfuracea extract, Evernia prunastri extract, farnesol, guaiac wood oil, hexyl cinnamal, hexyl salicylate, hydroxycitronellal, lavender oil, lime oil, linalyl acetate, mandarin oil, menthyl PCA, methylheptenone, nutmeg oil, rosemary oil, sweet orange oil, terpineol, tonka bean oil, triethyl citrate and/or vanillin.

In addition, the preparations according to the invention may contain the ingredients that are customary for cosmetic sunscreens, at the customary use concentrations.

### COMPARATIVE EXPERIMENT

The following preparations were produced and the color stability thereof was compared:

| | | | | | |
|---|---|---|---|---|---|
| | INCI | #1 | #2 | #3 | #4 |
| Fat phase | Caprylic/Capric Triglyceride | 2.00 | 2.00 | 2.00 | 2.00 |
| | Dicaprylyl Carbonate + Tocopherol | 20.00 | 20.00 | 20.00 | 20.00 |
| | C12-15 Alkyl Benzoate | 2.00 | 2.00 | 2.00 | 2.00 |
| | Diisostearoyl Polyglyceryl-3 Dimer | | | | |
| | Dilinoleate | | 2.00 | | 2.00 |
| | Butyl Methoxydibenzoylmethane | | 4.00 | 4.00 | 4.00 |
| Thickener | Hydroxypropyl Methylcellulose | 1.50 | 1.50 | 1.50 | 1.50 |
| Water phase | Iron(III) Chloride, Anhydrous | 0.00289 | | 0.00289 | 0.00289 |
| | Aqua | ad 100 | ad 100 | ad 100 | ad 100 |
| Alcohol phase phase | Alcohol Denat. + Aqua | 20.00 | 20.00 | 20.00 | 20.00 |

| | | | | | |
|---|---|---|---|---|---|
| | INCI | #5 | #6 | #7 | #8 |
| Fat phase | Caprylic/Capric Triglyceride | 2.00 | 2.00 | 2.00 | 2.00 |
| | Dicaprylyl Carbonate + Tocopherol | 20.00 | 20.00 | 20.00 | 20.00 |
| | C12-15 Alkyl Benzoate | 2.00 | 2.00 | 2.00 | 2.00 |
| | Diisostearoyl Polyglyceryl-3 Dimer | | | | |
| | Dilinoleate | | 2.00 | | 2.00 |
| | Diethylamino Hydroxybenzoyl Hexyl Benzoate | | 4.00 | 4.00 | 4.00 |
| Thickener | Hydroxypropyl Methylcellulose | 1.50 | 1.50 | 1.50 | 1.50 |
| Water phase | Iron(III) Chloride, Anhydrous | 0.00289 | | 0.00289 | 0.00289 |
| | Aqua | ad 100 | ad 100 | ad 100 | ad 100 |
| Alcohol phase | | | | | |
| phase | Alcohol Denat. + Aqua | 20.00 | 20.00 | 20.00 | 20.00 |

1) Heat fat phase to 75° C. until all components have melted.
2) Then add the thickener and mix thoroughly.
3) Stir the prepared water phase for 10 minutes before it is processed further.
4) Add the water phase (25° C.) to the warm fat phase (75° C.) with stirring and stir for 2-5 minutes until smooth.
5) Homogenize once.
6) After the homogenization, the alcohol phase is added at room temperature.
7) As soon as all components are distributed homogeneously, homogenization is performed for a second time.
8) Finally, stir for a further 5 minutes.
9) The samples were measured within 48 hours using the CIELAB Color System (see below).

### CIELAB COLOR SYSTEM

To determine the change in color of the emulsions, use was made of the CIELAB or CIE (L*a*b*) color space and an SP62 portable spectrophotometer from X-Rite Incorporated (Michigan, USA). A spectrophotometer measures spectral data, i.e. the amount of light energy reflected by an object in several intervals along the visible spectrum. The spectral data are illustrated as a spectral curve, which are transformed into the tristimulus space (CIEXYZ) by way of a computational transformation. ^{[1,2]} However, tristimulus values are only of limited use as color specifications, since they correlate poorly with visual attributes. Since the CIELAB color space measurement better describes the human perception of color ^{[3]}, this method was selected to evaluate the issue at hand. When a color is expressed in the CIELAB, L* defines the lightness, a* denotes the red/green value and b* the yellow/blue value. ^{[1,2]}

The spectrophotometer was controlled by an HP EliteDesk 800 G1 TWR (Intel^{®} Core™ i5vPro) computer that performed all color calculations from the digitized spectral data using the software Color iQC Professional with SLITaper^{®} Version 8.3.9 (X-Rite Incorporated, Michigan, USA). Each time the software was started, the device was properly calibrated with the supplied white and black standard.

Both the preparation and the measurement of all samples took place in a climate-controlled room (22 ± 1; 55 ± 5% rel. humidity). The samples were first prepared as follows:

A disposable petri dish made of polystyrene (35/10 mm) was filled, completely up to the edge and without bubbles, with the contents to be analyzed and was closed. The completely filled petri dish was then placed in a Puluz photobox (size: 22 x 23 x 24 cm) for the measurement.

In order to ensure that all samples are measured under the same standardized light conditions, a photobox (Morza PULUZ) having 20 white integrated LEDs was used. For the measurement, the spectrophotometer was placed on the lid of the Petri dish and activated. A total of 10 aleatoric measurements per emulsion were performed over the whole surface of the petri dish.

The major advantage of the CIELAB space is that it provides a means for measuring the differences between any two colors. The color difference thereof (ΔE) is calculated as the Euclidean distance of the L*a*b* values on the basis of Formula (1) below, and should be kept as small as possible: ^{[3]}

$$Δ E = \sqrt{{\left({L}_{2} - {L}_{1}\right)}^{2} + {\left({a}_{2} - {a}_{1}\right)}^{2} + {\left({b}_{2} - {b}_{1}\right)}^{2}}$$

### REFERENCES

1. Sharma, G. and Bala, R. (2017) Digital Color Imaging Handbook
2. X-Rite, I. A Guide to Understanding Color [Online]. Available at https://www.xrite.com/-/media/xrite/files/whitepaper_pdfs/110-001_a_guide_to_understanding_color_communication /110-001_understand_color_en.pdf
3. Weatherall, I. L. and Coombs, B. D. (1992) Skin color measurements in terms of CIELAB color space values, The Journal of investigative dermatology, vol. 99, no. 4
4. The BabelColor Company and Pascale, D. (2003) A comparison of four multimedia RGB spaces

Summary of the CIELab results of formula comparisons 1 to 8

| Comparison: | ΔE | Comparison: | ΔE | Description: |
|---|---|---|---|---|
| #1 with #2: | 5.96 | #5 with #6: | 8.48 | Color difference between Fe(III) and UVA filter + Diisostearoyl Polyglyceryl-3 Dimer Dilinoleate |
| #1 with #3: | 14.51 | #5 with #7: | 12.80 | Color difference between Fe(III) and UVA filter + Fe(III) |
| #1 with #4: | 5.15 | #5 with #8: | 7.19 | Color difference between Fe(III) and UVA filter + Diisostearoyl Polyglyceryl-3 Dimer Dilinoleate + Fe(III) |
| #2 with #3: | 18.87 | #6 with #7: | 14.89 | Color difference between UVA filter + Diisostearoyl Polyglyceryl-3 Dimer Dilinoleate and UVA filter + Fe(III) |
| #2 with #4: | 7.78 | #6 with #8: | 7.63 | Color difference between UVA filter + Diisostearoyl Polyglyceryl-3 Dimer Dilinoleate and UVA filter + Diisostearoyl Polyglyceryl-3 Dimer Dilinoleate + Fe(III) |
| #3 with #4: | 11.17 | #7 with #8: | 7.43 | Color difference between UVA filter + Fe(III) and UVA filter + Diisostearoyl Polyglyceryl-3 Dimer Dilinoleate + Fe(III) |

### EXAMPLES

The examples which follow are intended to illustrate the present invention without limiting it. Unless otherwise indicated, all amounts, proportions and percentages are based on the weight and the total amount or on the total weight of the preparations.

| | | | | |
|---|---|---|---|---|
| INCI | Example 1 | Example 2 | Example 3 | Example 4 |
| Octocrylene | 9.00 | | 9.00 | |
| Diisostearoyl Polyglyceryl-3-Dimer Dilinoleate | 2.00 | 2.50 | 1.50 | 1.00 |
| Carbomer | | | 0.2 | 0.1 |
| Acrylates/C 10-30 Alkyl Acrylate Crosspolymer | 0.1 | | | 0.15 |
| Cetyl Alcohol | 0.3 | | | |
| Perfume | 0.4 | 0.4 | 0.5 | 0.3 |
| Tocopheryl Acetate | 0.05 | 0.05 | 0.05 | 0.1 |
| Diethylamino Hydroxybenzoyl Hexyl Benzoate | | 0.5 | | |
| Hydroxyacetophenone | 0.4 | 0.4 | | |
| Behenyl Alcohol | | 0.9 | | |
| Tapioca Starch | | | 1.00 | |
| Myristyl Myristate | 1.00 | | | |
| Hydrogenated Rapeseed Oil | | 1.00 | | |
| Cellulose Gum | 0.01 | 0.5 | 0.01 | |
| Ethylhexylglycerin | 0.3 | | 0.15 | 0.3 |
| Dimethicone | | | 0.3 | 0.8 |
| Sodium Stearoyl Glutamate | | 0.25 | 0.3 | 0.3 |
| Silica Dimethyl Silylate | | 1.00 | 0.3 | |
| Xanthan Gum | 0.3 | 0.4 | 0.4 | 0.1 |
| Stearyl Alcohol | 0.5 | 0.9 | | |
| Sodium Hydroxide | 0.05 | | 0.05 | 0.09 |
| Glyceryl Stearate | | | 1 | 0.8 |
| Phenoxyethanol | | 0.5 | 0.3 | 0.6 |
| Copernicia Cerifera Cera | 0.5 | 0.5 | | |
| Distarch Phosphate | | | | 2.00 |
| Glyceryl Stearate Citrate | 2.50 | | | |
| Hydrogenated Coco-Glycerides | 1.00 | | 1.00 | |
| Caprylic/Capric Triglyceride | 2.00 | 2.00 | 2.00 | 2.00 |
| Ethylhexyl Cocoate | | | 2.00 | |
| Octyldodecanol | | | 2.00 | |
| Cetearyl Alcohol | | | 0.5 | 2.00 |
| Panthenol + Aqua | 1.4 | 1.4 | | |
| Phenylbenzimidazole Sulfonic Acid | | 0.5 | 1.00 | 2.00 |
| Dibutyl Adipate | | | | 2.00 |
| Titanium Dioxide | 3.00 | 2.00 | | |
| EDTA | 0.20 | 0.10 | 0 | 0.05 |
| Butylene Glycol Dicaprylate/Dicaprate | | 2.00 | | 3.00 |
| Ethylhexyl Triazone | | 2.00 | | 2.00 |
| Bis-Ethylhexyloxyphenol Methoxyphenyl Triazine | | 3.5 | 1.00 | 2.5 |
| C12-15 Alkyl Benzoate | 4.5 | 2.00 | 2.00 | 4.00 |
| Ethylhexyl Salicylate | 4.5 | 2.00 | 4.5 | 3.00 |
| Butyl Methoxydibenzoylmethane | 4.5 | 4.75 | 4.5 | 3.00 |
| Glycerin | 8.6 | 7.5 | 0.9 | 6.00 |
| Alcohol Denat. | 5.00 | 5.00 | 7.00 | 4.00 |
| Homosalate | 9.00 | 9.5 | 9.00 | |
| Perfumes | 0.005 | 0.002 | 0.1 | 0.05 |
| Aqua | To 100 | To 100 | To 100 | To 100 |

| | | |
|---|---|---|
| INCI | Example 5 | Example 6 |
| Ethylhexylglycerin | 0.30 | 0.30 |
| Dibutyl Adipate | 3.00 | 3.00 |
| Cetyl Palmitate | 4.00 | 2.00 |
| C12-15 Alkyl Benzoate | 5.00 | 5.00 |
| Caprylic/Capric Triglyceride | 2.00 | 2.00 |
| Butylene Glycol Dicaprylate/Dicaprate | 3.20 | 3.20 |
| Copernicia Cerifera Cera | 1.00 | 1.00 |
| Helianthus Annuus Seed Cera | 5.00 | 2.50 |
| Sucrose Polystearate + Hydrogenated Polyisobutene | 1.00 | 1.00 |
| Polyglyceryl-4 Diisostearate/Polyhydroxystearate/Sebacate | 0.25 | 0.25 |
| Sodium Stearoyl Glutamate | 0.40 | 0.40 |
| Glycerin | 5.00 | 5.00 |
| Phenoxyethanol | 0.50 | 0.50 |
| Cellulose Gum | 0.50 | 0.50 |
| Xanthan Gum | 0.15 | 0.15 |
| Microcrystalline Cellulose + Cellulose Gum | 1.00 | 1.00 |
| Alcohol Denat. | 5.00 | 5.00 |
| Diisostearoyl Polyglyceryl-3-Dimer Dilinoleate | 2.00 | 2.00 |
| EDTA | 0.10 | 0.00 |
| Ethylhexyl Salicylate | 3.30 | 3.30 |
| Bis-Ethylhexyloxyphenol Methoxyphenyl Triazine | 3.00 | 3.00 |
| Ethylhexyl Triazone | 1.50 | 1.50 |
| Butyl Methoxydibenzoylmethane | 3.00 | 3.00 |
| Phenylbenzimidazole Sulfonic Acid | 0.50 | 0.50 |
| Perfumes | 0.01 | 0.02 |
| Aqua | To 100 | To 100 |

1-16. (canceled)

17. A cosmetic preparation, wherein the preparation comprises
(a) 4-(tert-butyl)-4′-methoxydibenzoylmethane and/or hexyl 2-[4-(diethylamino)-2-hydroxybenzoyl]benzoate; and
(b) Diisostearoyl Polyglyceryl-3 Dimer Dilinoleate.

18. The preparation of claim 17, wherein the preparation is free of EDTA.

19. The preparation of claim 17, wherein the preparation is free of 3-(4-methylbenzylidene)camphor, 2-hydroxy-4-methoxybenzophenone, 2-ethylhexyl 4-methoxycinnamate, parabens, methylisothiazolinone, chloromethylisothiazolinone, DMDM hydantoin, polyethylene glycol ethers and polyethylene glycol esters.

20. The preparation of claim 17, wherein the preparation is free of octocrylene.

21. The preparation of claim 17, wherein the preparation comprises Diisostearoyl Polyglyceryl-3 Dimer Dilinoleate which has a viscosity of at least 1500 mPas in a 50% by weight solution in C12-15 alkyl benzoate at 25° C.

22. The preparation of claim 17, wherein the preparation comprises from 0.1% to 5% by weight Diisostearoyl Polyglyceryl-3 Dimer Dilinoleate, based on a total weight of the preparation.

23. The preparation of claim 17, wherein the preparation further comprises one or more iron-containing color pigments.

24. The preparation of claim 17, wherein the preparation is in the form of an O/W emulsion.

25. The preparation of claim 24, wherein the preparation comprises one or more O/W emulsifiers selected from glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate.

26. The preparation of claim 17, wherein the preparation further comprises one or more of 2,4-bis{[4-(2-ethylhexyloxy)-2-hydroxy]phenyl}-6-(4-methoxyphenyl)-1,3,5-triazine, 2,4,6-tris[anilino(p-carbo-2′-ethyl-1′-hexyloxy)]-1,3,5-triazine, 2-phenylbenzimidazole-5-sulfonic acid salt.

27. The preparation of claim 17, wherein the preparation comprises 4-(tert-butyl)-4′-methoxydibenzoylmethane and no hexyl 2-[4-(diethylamino)-2-hydroxybenzoyl]benzoate, and a weight ratio of 4-(tert-butyl)-4′-methoxydibenzoylmethane to Diisostearoyl Polyglyceryl-3 Dimer Dilinoleate in the preparation is from 1:50 to 1:1.

28. The preparation of claim 17, wherein the preparation contains hexyl 2-[4-(diethylamino)-2-hydroxybenzoyl]benzoate, and no 4-(tert-butyl)-4′-methoxydibenzoylmethane and a weight ratio of hexyl 2-[4-(diethylamino)-2-hydroxybenzoyl]benzoate to Diisostearoyl Polyglyceryl-3 Dimer Dilinoleate in the preparation is from 1:50 to 2:1.

29. The preparation of claim 17, wherein a weight ratio of (a) to (b) is from 1:25 to 3:1.

30. The preparation of claim 17, wherein the preparation further comprises one or more perfumes.

31. A method of producing a color-stable cosmetic preparation which comprises 4-(tert-butyl)-4′-methoxydibenzoylmethane and/or hexyl 2-[4-(diethylamino)-2-hydroxybenzoyl]benzoate, wherein the method comprises incorporating in the preparation Diisostearoyl Polyglyceryl-3 Dimer Dilinoleate in an amount which results in a color-stable preparation.

32. The method of claim 31, wherein the preparation is free of EDTA.

33. The method of claim 31, wherein the preparation is free of 3-(4-methylbenzylidene)camphor, 2-hydroxy-4-methoxybenzophenone, 2-ethylhexyl 4-methoxycinnamate, parabens, methylisothiazolinone, chloromethylisothiazolinone, DMDM hydantoin, polyethylene glycol ethers and polyethylene glycol esters.

34. The method of claim 31, wherein the preparation is free of octocrylene.

35. A method of preventing the formation of complexes of metal ions in a cosmetic preparation which comprises 4-(tert-butyl)-4′-methoxydibenzoylmethane and/or hexyl 2-[4-(diethylamino)-2-hydroxybenzoyl]benzoate and one or more metal ions, wherein the method comprises incorporating in the preparation Diisostearoyl Polyglyceryl-3 Dimer Dilinoleate in an amount which is sufficient to prevent the formation of metal complexes.

36. The method of claim 35, wherein the preparation is free of EDTA.
